(19) United States
(12) Patent Application Publication
(10) Pub. No.: US 2009/0306442 A1
(43) Pub. Date: Dec. 10, 2009

(54) OLIGOMERISATION CATALYST WITH PENDANT DONOR GROUPS

(75) Inventors: Marié Pretorius, Roodepoort (ZA); Annette Bollmann, Henley-on-Klip (ZA); Kevin Blann, Alberton (ZA); Matthew James Overett, Johannesburg (ZA)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: SASOL TECHNOLOGY (PTY) LIMITED, Johannesburg (ZA)

(21) Appl. No.: 12/302,742

(22) PCT Filed: May 28, 2007
(86) PCT No.: PCT/IB2007/052001

§ 371 Date: Aug. 10, 2009

(30) Foreign Application Priority Data
May 30, 2006 (ZA) 2006/04393

(51) Int. Cl. B01J 31/18; C07C 11/02; C07C 15/18; C07C 2/72; C07C 2/24

(52) U.S. Cl. 585/24; 502/155; 585/16; 585/428; 585/511; 585/513

(57) ABSTRACT

This invention relates to a process for producing an oligomeric product by the oligomerisation of at least one olefinic compound in the form of an olefin or a compound including a carbon to carbon double bond, by contacting the at least one olefinic compound with an oligomerisation catalyst which includes the combination of a source of a transition metal, and a ligating compound of the formula (R¹)ₘX¹(Y)X²(R²)ₙ. The invention also relates to an oligomerisation catalyst comprising the combination of a source of a transition metal, and a ligating compound of the formula (R¹)ₘX¹(Y)X²(R²)ₙ

### TECHNICAL FIELD

This invention relates to the oligomerisation of olefinic compounds in the presence of an oligomerisation catalyst which includes a ligating compound wherein at least one electron donating group thereon is linked through a linking moiety to a hetero atom of the ligating compound. The invention also relates to such an oligomerisation catalyst.

### BACKGROUND ART

A number of different oligomerisation technologies are known to produce α-olefins. Some of these processes, including the Shell Higher Olefins Process and Ziegler-type technologies, have been summarized in WO 04/056479 A1. The same document also discloses that the prior art (e.g. WO 03/053891 and WO 02/04119) teaches that chromium based catalysts containing heteroaromatic ligands with both phosphorus and nitrogen hetero atoms, selectively catalyse the trimerisation of ethylene to 1-hexene.

Processes wherein transition metals and heteroatomic ligands are combined to form catalysts for trimerisation, tetramerisation, oligomerisation and polymerisation of olefinic compounds have also been described in different patent applications such as WO 03/053890 A1; WO 03/053891; WO 04/056479 A1; WO 04/056477 A1; WO 04/056480 A1; WO 04/056478 A1; South African provisional patent application number 2004/3805; South African provisional patent application number 2004/4839; South African provisional patent application number 2004/4841; and UK provisional patent application no. 0520085.2; and U.S. provisional patent application No. 60/760,928.

It has now been found that when an olefinic compound is oligomerised in the presence of an oligomerisation catalyst which includes a ligating compound wherein at least one electron donating group thereon is linked through a linking moiety to a hetero atom of the ligating compound, the selectivity of the process is influenced, for example to provide a high selectivity towards a trimerised product instead of a tetramerised product. Good selectivity towards linear alpha olefin products was also achieved. This is illustrated by comparing example 3 to comparative example 1.

Organometallics 2002, 21, 5122-5135 discloses titanium based catalysts for the trimerisation of ethylene 35 to 1-hexene. The cyclopentadienyl ligands disclosed include pendant arene groups thereon which bind to the titanium. However the disclosed ligands do not have electron donating groups linked through a linking moiety to a hetero atom of the ligand and are very different to the ligands of the present invention.

Journal of Organometallic Chemistry 690 (2005) 713-721 discloses chromium complexes of tridentate imine ligands I and amine ligands II:

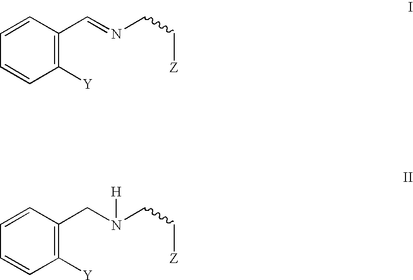

In each case Y was an electron donating heteroatomic (that is containing an atom other than H and C) group such as PPh₂, SMe or OMe; and Z was also a heteroatomic (that is containing a compound other than H or C) group such as PPh₂, SEt, C₅H₄N, NMe₂, OMe or SMe. In the chromium complexes formed with these ligands, the hetero atoms in Y and Z, as well as N in the ligands I and II formed bonds with the chromium atom.

Most surprising it has now been found that a heteroatomic group in the form of Y in ligands I and II is not required to provide an effective trimerisation catalyst. The omission of such a Y group in such and similar ligands has the advantage that in at least some cases it may lead to high selectivities to 1-hexene and/or alpha olefinic compounds and/or, high reaction rates and/or good catalyst stability.

### DISCLOSURE OF THE INVENTION

According to the present invention there is provided a process for producing an oligomeric product by the oligomerisation of at least one olefinic compound by contacting the at least one olefinic compound with an oligomerisation catalyst which includes the combination of

i) a source of a transition metal; and
ii) a ligating compound of the formula

(R¹)ₘX¹(Y)X²(R²)ₙ
wherein
   X¹ and X² are independently an atom selected from the group consisting of N, P, As, Sb, Bi, O, S and Se or said atom oxidized by S, Se, N or O, where the valence of X¹ and/or X² allows for such oxidation;
   Y is a linking group between X¹ and X²;
   m and n are independently 0, 1 or a larger integer; and
   R¹ and R² are independently selected from the group consisting of hydrogen, a hydrocarbyl group, a heterohydrocarbyl group, and an organoheteryl group; R¹ being the same or different when m>1; R² being the same or different when n>1; and at least one R¹ or R² is a moiety of formula

(L)(D)
wherein: L is a linking moiety between X¹ or X² and D; and
   D is an electron donating moiety which includes at least one multiple bond between adjacent atoms which multiple bond renders D capable of bonding with the transition metal in the source of transition metal; provided that when D is a moiety derived from an aromatic compound with a ring atom of the aromatic compound bound to L, D has no electron donating moiety that is bound to a ring atom of the aromatic compound adjacent to the ring atom bound to L, and that is in the form of a heterohydrocarbyl group, a heterohydrocarbylene group, a heterohydrocarbylidene group, or an organoheteryl group that is capable of bonding by a coordinate covalent bond to the transition metal in the source of transition metal.

An electron donating moiety is defined in this specification as a moiety that donates electrons used in chemical bond, including coordinate covalent bond, formation.

In this specification the following further definitions also apply:

a hydrocarbyl group is a univalent group formed by removing one hydrogen atom from a hydrocarbon;
a hydrocarbylene group is a divalent group formed by removing two hydrogen atoms from the same or different carbon atoms in a hydrocarbon, the resultant free valencies of which are not engaged in a double bond;
a hydrocarbylidene group is a divalent group formed by removing two hydrogen atoms from the same carbon atom of a hydrocarbon, the resultant free valencies of which are engaged in a double bond;
a heterohydrocarbyl group is a univalent group formed by removing one hydrogen atom from a heterohydrocarbon, that is a hydrocarbon compound which includes at least one hetero atom (that is, not being H or C), and which group binds with other moieties through the resultant free valency on that carbon atom;
a heterohydrocarbylene group is a divalent group formed by removing two hydrogen atoms from the same or different carbon atoms in a heterohydrocarbon, the free valencies of which are not engaged in a double bond and which group binds with other moieties through the resultant free valencies on that or those carbon atoms;
a heterohydrocarbylidene group is a divalent group formed by removing two hydrogen atoms from the same carbon atom of a heterohydrocarbon, the free valencies of which are engaged in a double bond;
an organoheteryl group is a univalent group containing carbon atoms and at least one hetero atom, and which has its free valence at an atom other than carbon; and
olefinic compound is an olefin or a compound including a carbon to carbon double bond, and olefinic moiety has corresponding meaning.

### Oligomeric Product

The oligomeric product may be an olefin, or a compound including an olefinic moiety. Preferably the oligomeric product includes an olefin, more preferably an olefin containing a single carbon-carbon double bond, and preferably it includes an α-olefin. The olefin product may include hexene, preferably 1-hexene, alternatively or additionally it includes octene, preferably 1-octene. In a preferred embodiment of the invention the olefinic product includes hexene, preferably 1-hexene.

In one preferred embodiment of the invention the oligomerisation process is a selective process to produce an oligomeric product containing more than 30% by mass of total product of a single olefin product. The olefin product may be hexene, preferably 1-hexene.

Preferably the product contains at least 35% of the said olefin, preferably α-olefin, but it may be more than 40%, 50%, 60% or even 80% and 90% by mass. Preferably the product contains less than 30% and even less than 10% by mass of another olefin.

The olefin being present in more than 30% by mass of the total product may comprise more than 80%, preferably more than 90%, preferably more than 95% by mass of an α-olefin.

The olefinic product may be branched, but preferably it is non-branched.

### Oligomerisation

Preferably the oligomerisation process comprises a trimerisation process.

The process may be oligomerisation of two or more different olefinic compounds to produce an oligomer containing the reaction product of the two or more different olefinic compounds. Preferably however, the oligomerisation (preferably trimerisation) comprises the oligomerisation of a single monomer olefinic compound.

In one preferred embodiment of the invention the oligomerisation process is oligomerisation of a single α-olefin to produce an oligomeric α-olefin. Preferably it comprises the trimerisation of ethylene, preferably to 1-hexene.

### Olefinic Compound to be Oligomerised

The olefinic compound may comprise a single olefinic compound or a mixture of olefinic compounds. In one embodiment of the invention it may comprise a single olefin.

The olefin may include multiple carbon-carbon double bonds, but preferably it comprises a single carbon-carbon double bond. The olefin may comprise an α-olefin with 2 to 30 carbon atoms, preferably 2 to 10 carbon atoms. The olefinic compound may be selected from the group consisting of ethylene, propene, 1-butene, 1-pentene, 1-hexene, 1-heptene, and 1-octene, 1-nonene, 1-decene, 3-methyl-1-butene, 3-methyl-1-pentene, 4-methyl-1-pentene, styrene, p-methyl styrene, 1-dodecene or combinations thereof. Preferably it comprises ethylene or propene, preferably ethylene. The ethylene may be used to produce hexene, preferably 1-hexene.

### Oligomerisation Catalyst

### Activator

In a preferred embodiment of the invention the catalyst also includes one or more activators. Such an activator may be a compound that generates an active catalyst when the activator is combined with the source of transition metal and the ligating compound.

Suitable activators include aluminium compounds, boron compounds, organic salts, such as methyl lithium and methyl magnesium bromide, inorganic acids and salts, such a tetrafluoroboric acid etherate, silver tetrafluoroborate, sodium hexafluoroantimonate and the like.

Suitable aluminium compounds include compounds of the formula Al(R⁹)₃ (R⁹ being the same or different), where each R⁹ is independently a C₁-C₁₂ alkyl, an oxygen containing moiety or a halide, aluminoxanes, and compounds such as LiAlH₄ and the like. Aluminoxanes are well known in the art as typically oligomeric compounds which can be prepared by the controlled addition of water to an alkylaluminium compound, for example trimethylaluminium. Such compounds can be linear, cyclic, cages or mixtures thereof. Examples of suitable aluminium compounds in the form of organoaluminium activators include trimethylaluminium (TMA), triethylaluminium (TEA), tri-isobutylaluminium (TIBA), tri-n-octylaluminium, methylaluminium dichloride, ethylaluminium dischloride, dimethylaluminium chloride, diethylaluminium chloride, aluminium isopropoxide, ethylaluminiumsesquichloride, methylaluminiumsesquichloride, methylaluminoxane (MAO), ethylaluminoxane (EAO), isobuthylaluminoxane (iBuAO), modified alkylaluminoxanes such as modified methylaluminoxane (MMAO) and mixture thereof.

Examples of suitable boron compounds are boroxines, NaBH₄, triethylborane, tris(pentafluorophenyl)borane, trityl tetrakis(pentafluorophenyl)borate, dimethylanilinium tetrakis(pentafluorophenyl)borate, tributyl borate and the like.

The activator may be a compound as described in UK Provisional Patent Application No. 0520085.2 which is incorporated herein by reference.

The activator may also be or contain a compound that acts as a reducing or oxidizing agent, such as sodium or zinc metal and the like, or hydrogen or oxygen and the like.

The activator may be selected from alkylaluminoxanes such as methylaluminoxane (MAO), high stability methylaluminoxane (MAO HS), modified alkylaluminoxanes such as modified methylaluminoxane (MMAO). MMAO is described later in this specification.

The transition metal source and the aluminoxane may be combined in proportions to provide Al/transition metal molar ratios from about 1:1 to 10 000:1, preferably from about 1:1 to 1500:1, and more preferably from 1:1 to 1000:1.

The oligomerisation process may also include the step of the continuous addition of the activator, including a reducing (such as hydrogen (H₂)) or oxidizing agent, to a solution containing the transition metal source.

It should be noted that aluminoxanes generally also contain considerable quantities of the corresponding trialkylaluminium compounds used in their preparation. The presence of these trialkylaluminium compounds in aluminoxanes can be attributed to their incomplete hydrolysis with water.

It has been found that modified methylaluminoxane (MMAO) is especially suitable as an activator which may result in improved activity and stability of the catalyst.

MMAO is methyl aluminoxane wherein one or more, but not all methyl groups have been replaced by one or more non-methyl moieties. Preferably the non-methyl moiety is an organyl, preferably a hydrocarbyl or a heterohydrocarbyl. Preferably it is an alkyl, preferably isobutyl or n-octyl.

### Source of Transition Metal

Preferably the source of transition metal as set out in (i) above is a source of a Group 4B to 6B transition metal. Preferably it is a source of Cr, Ti, V, Ta or Zr, more preferably Cr, Ti, V or Ta. Preferably it is a source of either Cr, Ta or Ti. Most preferably it is a source of Cr.

The source of the Group 4B to 6B transition metal may be an inorganic salt, an organic salt, a coordination compound or an organometallic complex.

Preferably the source of transition metal is a source of chromium and preferably it is selected from the group consisting of chromium trichloride tris-tetrahydrofuran; (benzene)tricarbonyl chromium; chromium (III) octanoate; chromium (III) hexaonate; chromium hexacarbonyl; chromium (III) acetylacetonate, chromium (III) naphthenate, chromium (III) 2-ethylhexanoate. Preferably it is chromium (III) acetylacetonate.

### Ligating Compound

As stated above at least one R¹ or R² is a moiety of the formula

(L)(D)
wherein: L is a linking moiety between X¹ or X² and D; and
   D is an electron donating moiety which includes at least one multiple bond between adjacent atoms which multiple bond renders D capable of bonding with the transition metal in the source of transition metal; provided that when D is a moiety derived from an aromatic compound with a ring atom of the aromatic compound bound to L, D has no electron donating moiety that is bound to a ring atom of the aromatic compound adjacent to the ring atom bound to L, and that is in the form of a heterocarbyl group, a heterohydrocarbylene group, a heterohydrocarbylidene group, or an organoheteryl group that is capable of bonding by a coordinate covalent bond to the transition metal in the source of transition metal.

Preferably D is an electron donating moiety capable of bonding with the transition metal by a coordinate covalent bond.

Preferably, when D is an aromatic compound with a ring atom of the aromatic compound bound to L, D has no electron donating moiety in any form capable of bonding by a coordinate covalent bond to the transition metal bound to a ring atom of the aromatic compound adjacent to the ring atom bound to L.

Preferably D is an electron donating moiety in the form of a hydrocarbyl moiety or a heterohydrocarbyl moiety which includes at least one multiple bond between adjacent atoms, preferably adjacent carbon atoms, wherein at least one such multiple bond renders D capable of bonding by a coordinate covalent bond to the transition metal. Preferably D is a hydrocarbyl moiety.

D may be an aromatic or heteroaromatic moiety. D may include a moiety (including a hydrocarbyl or heterohydrocarbyl) other than H bound to a ring atom defined by D. D may include one or more electron donating moieties. Preferably D has no such electron donating moiety, preferably no moiety other than H, as a non-ring atom bound to a ring atom defined by D. Preferably D is an aromatic moiety.

In one embodiment of the invention D may comprise phenyl, or a substituted phenyl wherein one or more moieties other than H are bound as a non-ring atom to a ring atom of D.

Preferably D is an aromatic or heteroaromatic moiety selected from the group consisting of phenyl, naphthyl, 7-(1,2,3,4-tetrahydronaphthyl), anthracenyl, phenanthrenyl, phenalenyl, 3-pyridyl, 3-thiopeneyl, 7-benzofuranyl, 7-(2H-1-benzopyranyl), 7-quinolinyl and 6-benzisoxazolyl.

L is preferably bound to a single atom of D, preferably to a single ring atom of D where D is an aromatic or a heteroaromatic moiety. Preferably L is bound to D by means of a single bond. Preferably L is bound to an atom (preferably a carbon atom) of D which atom of D is linked to another atom of D (preferably a carbon atom) by means of a multiple bond. Preferably L is bound to a ring atom of D where D is an aromatic or a heteroaromatic moiety.

L may be bound to X¹ or X² by means of a single bond or a double bond.

Preferably L is aliphatic and preferably L includes no multiple bonds between atoms in the L moiety. Preferably L includes not more than 3 carbon atoms, and all the carbon atoms of L may be sp³ carbon atoms. Preferably L is a hydrocarbon moiety. In one embodiment of the invention L may include one or more carbon atoms where all carbon atoms only have saturated bonds, and preferably L is —CH₂—. Alternatively L may comprise one or more carbon atoms with unsaturated bonds, and L may be ═CH—.

L may be selected from —CH₂—, —CH═, —CH₂—CH₂—, —CH═CH—, —CH₂—CH₂—CH₂—, —CH═CH—CH₂—, —CH₂—CH═CH—, —CH(CH₃)—CH₂—CH₂—, —CH₂—CH(CH₃)—CH₂—, —CH₂—CH₂—CH(CH₃)— and —CH₂—C(CH₃)₂—CH₂—.

Combined (L)(D) may be a moiety selected from benzyl, ethyl-phenyl, propyl-phenyl, methyl-naphthyl, ethyl-naphthyl, propyl-naphthyl, methyl-anthracenyl, methyl-phenanthrenyl, methyl-phenalenyl, methyl-3-(pyridyl), methyl-3-(thiopeneyl), methyl-7-(benzofuranyl), methyl-7-(2H-1-benzopyranyl), methyl-7-(quinolinyl) and methyl-6-(benzisoxazolyl).

Y may be selected from the group consisting of an organic linking group such as a hydrocarbylene, substituted hydrocarbylene, heterohydrocarbylene and a substituted heterohydrocarbylene; an inorganic linking group comprising either a single- or two-atom linker spacer; and a group comprising methylene; dimethylmethylene; ethylene; ethene-1,2-diyl; propane-1,2-diyl, propane-1,3-diyl; cyclopropane-1,1-diyl; cyclopropane-1,2-diyl; cyclobutane-1,2-diyl, cyclopentane-1,2-diyl, cyclohexane-1,2-diyl, cyclohexane-1,1-diyl; 1,2-phenylene; naphthalene-1,8-diyl; phenanthrene-9,10-diyl, phenanthrene-4,5-diyl, 1,2-catecholate, 1,2-diarylhydrazine-1,2-diyl (—N(Ar)—N(Ar)—) where Ar is an aryl group; 1,2-dialkylhydrazine-1,2-diyl (—N(Alk)-N(Alk)-) where Alk is an alkyl group; —B(R⁷)—, —Si(R⁷)₂—, —P(R⁷)— and —N(R⁷)— where R⁷ is hydrogen, a hydrocarbyl or heterocarbyl or halogen. Preferably, Y may be —N(R⁷)— and R⁷ may be selected from the group consisting of hydrogen, alkyl, substituted alkyl, aryl, substituted aryl, aryloxy, substituted aryloxy, halogen, alkoxycarbonyl, carbonyloxy, alkoxy, aminocarbonyl, carbonylamino, dialkylamino, silyl groups or derivatives thereof, and aryl substituted with any of these substituents. Preferably R⁷ may be a hydrocarbyl or a heterohydrocarbyl or an organoheteryl group. R⁷ may be methyl, ethyl, propyl, isopropyl, cyclopropyl, allyl, butyl, tertiary-butyl, sec-butyl, cyclobutyl, pentyl, isopentyl, 1,2-dimethylpropyl (3-methyl-2-butyl), 1,2,2-trimethylpropyl (R/S-3,3-dimethyl-2-butyl), 1-(1-methylcyclopropyl)-ethyl, neopentyl, cyclopentyl, cyclohexyl, cycloheptyl, cyclo-octyl, decyl, cyclodecyl, 1,5-dimethylheptyl, 2-naphthylethyl, 1-naphthylmethyl, adamantylmethyl, 1-adamantyl, 2-adamantyl, 2-isopropylcyclohexyl, 2,6-dimethylcyclohexyl, cyclododecyl, 2-methylcyclohexyl, 3-methylcyclohexyl, 4-methylcyclohexyl, 2-ethylcyclohexyl, 2-isopropylcyclohexyl, 2,6-dimethyl-cyclohexyl, exo-2-norbornanyl, isopinocamphenyl, dimethylamino, phthalimido, pyrrolyl, trimethylsilyl, dimethyl-tertiary-butylsilyl, 3-trimethoxylsilane-propyl, indanyl, cyclohexanemethyl, 2-methoxyphenyl, 3-methoxyphenyl, 4-methoxyphenyl, 4-tertiary-butylphenyl, 4-nitrophenyl, (1,1′-bis(cyclohexyl)-4,4′-methylene), 1,6-hexylene, 1-naphthyl, 2-naphthyl, N-morpholine, diphenylmethyl, 1,2-diphenyl-ethyl, phenylethyl, 2-methylphenyl, 3-methylphenyl, 4-methylphenyl, 2,6-dimethyl-phenyl, 1,2,3,4-tetrahydronaphthyl, or a 2-octyl group.

Preferably Y includes at least two, and preferably only two atoms in the shortest link between X¹ and X². The said two atoms may form part of a cyclic structure, alternatively they form part of an acyclic structure.

In one embodiment of the invention Y is a moiety of formula

—Y¹—Y²
wherein: Y¹ and Y² are independently CR₂¹⁹ or AR²⁰, wherein R¹⁹ and R²⁰ are independently hydrogen, a hydrocarbyl group or a heterocyclocarbyl group, and A is selected from the group consisting of N, P, As, Sb and Bi. Preferably A is N. It will be appreciated that in CR₂¹⁹, R₁₉ can be the same or different.

Preferably R¹⁹ and R²⁰ are independently H or a hydrocarbyl group, preferably an alkyl.

Preferably Y¹ and Y² are the same. In one embodiment of the invention Y may be

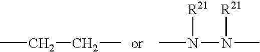

wherein each R²¹ is independently a hydrocarbyl group, preferably an alkyl group.

In an alternative embodiment of the invention Y may comprise a moiety derived from a cyclic compound wherein two atoms of the cyclic ring structure are bond to X¹ and X² respectively. The moiety derived from a cyclic compound may comprise a moiety derived from a cyclic organic compound which may include at least one heteroatom (that is an atom other than H and C). Preferably the cyclic compound comprises an aromatic compound or a heteroaromatic compound. Preferably it comprises an aromatic compound and in one embodiment, adjacent carbon ring atoms are bound to X¹ and X² respectively. Preferably Y comprises a moiety derived from a monocyclic aromatic compound, preferably a benzene ring with adjacent ring atoms bound to X¹ and X² respectively.

X¹ and/or X² may be a potential electron donor for coordination with the transition metal referred to in (i).

X¹ and/or X², may be independently oxidised by S, Se, N or O.

It will be appreciated that m and n are dependent on factors such as the valence and oxidation state of X¹ and X², bond formation of Y with X¹ and X² respectively, and bond formation of R¹ and R² with X¹ and X² respectively. Preferably both m and n are not O.

In one embodiment of the invention the ligating compound may be of the formula

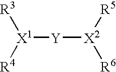

wherein Y is a linking group between X¹ and X²; X¹ and X² are independently selected from the group consisting of N, P, As, Sb and Bi; and R³ to R⁶ are each independently hydrogen, a hydrocarbyl group or a heterohydrocarbyl group and at least one of R³ to R⁶ is a moiety of formula

(L)(D)
wherein:
   L is a linking moiety between X¹ or X² and D; and
   D is an electron donating moiety which includes at least one multiple bond between adjacent atoms which multiple bond renders D capable of bonding with the transition metal in the source of transition metal;
   provided that when D is an aromatic compound with a ring atom of the aromatic compound bound to L, D has no electron donating moiety that is bound to a ring atom of the aromatic compound adjacent to the ring atom bound to L and that is in the form of a heterohydrocarbyl group, a heterohydrocarbylene group, a heterohydrocarbylidene group, or an organoheteryl group that is capable of bonding by a coordinate covalent bond to the transition metal in the source of transition metal.

Any of R³ to R⁶ which is not a moiety of formula (L)(D) may be an aromatic or heteroaromatic moiety. The aromatic or heteroaromatic moiety may include one or more substituents other than H on one or more aromatic carbon atoms, but preferably no such substituents are provided.

Preferably at least two, preferably all of R³ to R⁶ are moieties of formula (L)(D) as defined above.

Preferably L and D are as defined above.

Preferably X¹ or X² are the same and preferably both are P.

Preferably Y is as defined above and preferably Y is a moiety of formula —Y¹—Y² as defined above.

In an alternative embodiment of the invention the ligating compound may be of formula

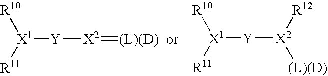

wherein: Y is as defined above; (L)(D) is as defined above; X¹ or X² are independently selected from the group consisting of N, P, As, Sb and Bi; R¹⁰ to R¹² are each independently hydrogen, a hydrocarbyl group or a heterohydrocarbyl group.

Preferably R¹² is hydrogen.

Preferably Y is as defined above.

Preferably X¹ and X² are different. Preferably X² is N and preferably X¹ is P.

Preferably =(L)(D) is

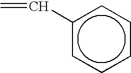

and -(L)(D) is benzyl

R¹⁰ and R¹¹ may each be a hydrocarbyl or heterohydrocarbyl moiety. Preferably each of R³ to R⁶, R¹⁰ and R¹¹ is an aromatic or heteroaromatic moiety, more preferably an aromatic moiety. The aromatic or heteroaromatic moiety may include one or more substituents other than H on one or more aromatic carbon atoms, but preferably no such substituents are provided. The aromatic moiety may comprise phenyl or a substituted phenyl.

Non-limiting examples of the ligating compound are (benzyl)₂PN(methyl)N(methyl)P(benzyl)₂;
(benzyl)₂PN(ethyl)N(ethyl)P(benzyl)₂;
(benzyl)₂PN(i-propyl)N(i-propyl)P(benzyl)₂;
(benzyl)₂PN(methyl)N(ethyl)P(benzyl)₂;
(benzyl)₂PN(methyl)N(i-propyl)P(benzyl)₂;
(benzyl)₂PN(methyl)N(t-butyl)P(benzyl)₂;
(benzyl)₂PCH₂N(i-propyl)P(benzyl)₂;
(allyl)₂PN(ethyl)N(ethyl)P(allyl)₂;
(phenyl)₂P—C₂H₄—N═C(H)-phenyl;
(phenyl)₂P—C₂H₄—N(H)—CH₂-phenyl;
(benzyl)(phenyl)PN(ethyl)N(ethyl)P(benzyl)(phenyl);
(benzyl)(phenyl)PN(ethyl)N(ethyl)P(phenyl)₂;
(benzyl)(phenyl)PN(ethyl)N(ethyl)P(benzyl)₂;
(ethyl-phenyl)₂PN(ethyl)N(ethyl)P(ethyl-phenyl)₂;
(propyl-phenyl)₂PN(ethyl)N(ethyl)P(propyl-phenyl)₂;
(methyl-naphthyl)₂PN(ethyl)N(ethyl)P(methyl-naphthyl)₂;
(ethyl-naphthyl)₂PN(ethyl)N(ethyl)P(ethyl-naphthyl)₂;
(benzyl)₂PN(isopropyl)P(benzyl)₂;
(benzyl)₂PN(methyl)P(benzyl)₂;
(benzyl)₂PN(ethyl)P(benzyl)₂;
(benzyl)₂PN(1,2-dimethylpropyl)P(benzyl)₂;
(benzyl)₂P-ethene-1,2-diyl-P(benzyl)₂;
(benzyl)₂P-ethylene-P(benzyl)₂;
(benzyl)₂P-1,2-phenylene-P(benzyl)₂.

The ligating compound may include a polymeric moiety to render the reaction product of the source of transition metal and the said ligating compound to be soluble at higher temperatures and insoluble at lower temperatures e.g. 25° C. This approach may enable the recovery of the complex from the reaction mixture for re-use and has been used for other catalyst as described by D. E. Bergbreiter et al., J. Am. Chem. Soc., 1987, 109, 177-179. In a similar vein these transition metal catalysts can also be immobilised by binding the ligating compound to silica, silica gel, polysiloxane or alumina backbone as, for example, demonstrated by C. Yuanyin et al., Chinese J. React. Pol., 1992, 1(2), 152-159 for immobilising platinum complexes.

The ligating compound may include multiple ligating units or derivatives thereof.

The ligating compounds may be prepared using procedures known to one skilled in the art and procedures forming part of the state of the art.

The oligomerisation catalyst may be prepared in situ, that is in the reaction mixture in which the oligomerisation reaction is to take place. Typically the oligomerisation catalyst will be prepared in situ. However it is foreseen that the catalyst may be pre-formed or partly pre-formed.

The source of transition metal and ligating compound may be combined (in situ or ex situ) to provide any suitable molar ratio, preferably a transition metal to ligand compound molar ratio, from about 0.01:100 to 000:1, preferably from about 0.1:1 to 10:1.

During catalyst preparation, the transition metal may be present in a range from 0.01 micromol to 200 mmol/l, preferably from 1 micromol to 15 mmol/l.

The process may also include combining one or more different sources of transition metal with one or more different ligating compounds.

The oligomerisation catalyst or its individual components, in accordance with the invention, may also be immobilised by supporting it on a support material, for example, silica, alumina, MgCl₂, zirconia, artificial hectorite or smectite clays such as Laponite™ RD or mixtures thereof, or on a polymer, for example polyethylene, polypropylene, polystyrene, or poly(aminostyrene). The catalyst can be formed in situ in the presence of the support material, or the support can be pre-impregnated or premixed, simultaneously or sequentially, with one or more of the catalyst components or the oligomerisation catalyst. In some cases, the support material can also act as a component of the activator. This approach would also facilitate the recovery of the catalyst from the reaction mixture for reuse.

### Process

The olefinic compound or mixture thereof to be oligomerised according to this invention can be introduced into the process in a continuous or batch fashion.

The olefinic compound or mixture of olefinic compounds may be contacted with the catalysts at a pressure of 100 kPa or higher, preferably greater than 1000 kPa, more preferably greater than 3000 kPa. Preferred pressure ranges are from 1000 to 30 000 kPa, more preferably from 3000 to 10 000 kPa.

The process may be carried out at temperatures from −100° C. to 250° C. Temperatures in the range of 15-150° C. are preferred. Particularly preferred temperatures range from 35-120° C.

The reaction products derived from the reaction as described herein, may be prepared using the disclosed catalysts by a homogeneous liquid phase reaction in the presence or absence of an inert solvent, and/or by slurry reaction where the catalysts and the oligomeric product is in a form that displays little or no solubility, and/or a two-phase liquid/liquid reaction, and/or a bulk phase reaction in which neat reagent and/or product olefins serve as the dominant medium, and/or gas phase reaction, using conventional equipment and contacting techniques.

The reaction may also be carried out in an inert solvent. Any inert solvent that does not react with the activator can be used. These inert solvents may include any saturated aliphatic and unsaturated aliphatic and aromatic hydrocarbon and halogenated hydrocarbon. Typical solvents include, but are not limited to, benzene, toluene, xylene, cumene, heptane, methylcyclohexane, methylcyclopentane, cyclohexane, Isopar C, Isopar E, Isopar H, Norpar, as well as the product formed during the reaction in a liquid state and the like.

The reaction may be carried out in a plant which includes reactor types known in the art. Examples of such reactors include, but are not limited to, batch reactors, semi-batch reactors and continuous reactors. The plant may include, in combination a) a stirred or fluidised bed reactor system, b) at least one inlet line into this reactor for olefin reactant and the catalyst system, c) effluent lines from this reactor for oligomerisation reaction products, and d) at least one separator to separate the desired oligomerisation reaction products which may include a recycle loop for solvents and/or reactants and/or products which may also serve as temperature control mechanism.

According to another aspect of the present invention there is provided an oligomerisation product prepared by a process substantially as described hereinabove.

According to yet another aspect of the present invention there is provided an oligomerisation catalyst which includes the combination of

i) a source of a transition metal; and
ii) a ligating compound of the formula

(R¹)ₘX¹(Y)X²(R²)ₙ
wherein:
   X¹ and X² are independently selected from the group consisting of N, P, As, Sb, Bi, O, S and Se;
   Y is a linking group between X¹ and X²;
   m and n are independently 0, 1 or a larger integer; and
   R¹ and R² are independently hydrogen, a hydrocarbyl group or a heterohydrocarbyl group; R¹ being the same or different when m>1; R² being the same or different when n>1; and at least one R¹ or R² is a moiety of formula

(L)(D)
wherein: L is a linking moiety between X¹ or X² and D; and
   D is an electron donating moiety which includes at least one multiple bond between adjacent atoms which multiple bond renders D capable of bonding with the transition metal in the source of transition metal; provided that when D is a moiety derived from an aromatic compound with a ring atom of the aromatic compound bound to L, D has no electron donating moiety that is bound to a ring atom of the aromatic compound adjacent to the ring atom bound to L, and that is in the form of a heterohydrocarbyl group, a heterohydrocarbylene group, a heterohydrocarbylidene group, or an organoheteryl group that is capable of bonding by a coordinate covalent bond to the transition metal in the source of transition metal.

The catalyst may also further include an activator as set out above.

The catalyst may comprise a trimerisation catalyst.

### EXAMPLES OF THE INVENTION

The invention will now be further described by means of the following non-limiting comparative examples and examples according to the invention in which the ligands set out below are used and which demonstrate the shift of selectivity to hexene brought about by the invention:

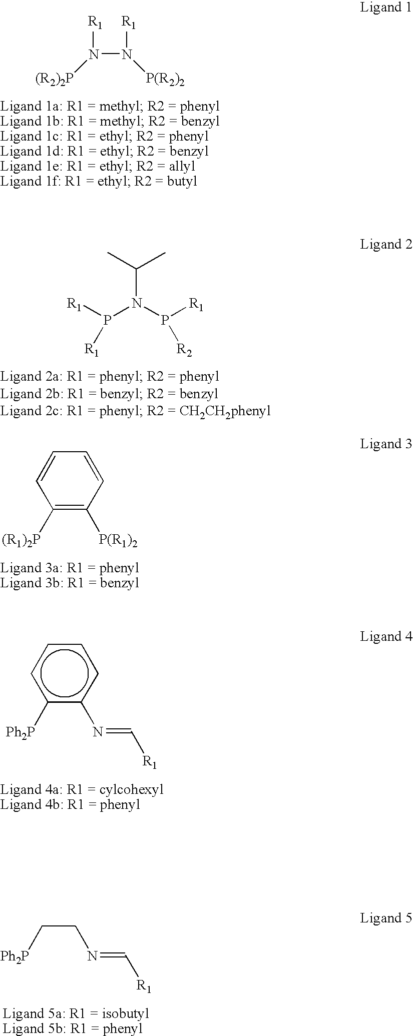

### Synthesis of Ligands

All ligands were prepared by procedures similar to those reported in literature. References include, amongst others: Slawin, A. M. Z; Wainwright, M and Woollins, J. D.; J. Chem. Soc., Dalton Trans. 2002, 513-519; Blann, K.; Bollmann, A.; Dixon, J. T., et al. Chem. Commun., 2005, 620-621; Dennett, J. N. L.; Gillon, A. L.; Pringle, P. G. et al. Organometallics; 2004; 23, 6077-6079; Doherty, S.; Knight, J. G.; Scanlan, T. H. et al, Journal of Organometallic Chemistry, 2002, 650, 231.

### Comparative Example 1 (Relative to Example 2)

### Ethylene Oligomerisation Reaction Using Cr(acetylacetonate)₃, (phenyl)₂PN(methyl)N(methyl)P(phenyl)₂ (Ligand 1a) and MMAO-3A in Methylcyclohexane at 60° C./4500 kPa

A solution of 1.07 mg of (phenyl)₂PN(methyl)N(methyl)P(phenyl)₂ (2.5 μmol) in 1.0 ml of methylcyclohexane was added to a solution of 0.88 mg chromium(acetylacetonate)₃ (2.5 μmol) in 1.0 ml of methylcyclohexane in a Schlenk tube. MMAO-3A (modified methylaluminoxane, 2.4 mmol) was added to this solution. This mixture was then transferred to a 450 ml pressure reactor (autoclave) containing of methylcyclohexane (100 ml) at 55° C. The autoclave was charged with ethylene after which the reactor temperature was controlled at 60° C. while the ethylene pressure was maintained at 4500 kPa. The reaction was terminated after 38 min, by discontinuing the ethylene feed to the reactor and cooling the reactor to below 20° C. After releasing the excess ethylene from the autoclave, the liquid contained in the autoclave was quenched with ethanol followed by 10% hydrochloric acid in water. Nonane was added as an internal standard for the analysis of the liquid phase by GC-FID. A small sample of the organic layer was dried over anhydrous sodium sulfate and then analysed by GC-FID. The remainder of the organic layer was filtered to isolate the solid products. These solid products were dried overnight in an oven at 100° C. and then weighed. The total product mass was 116.46 g. The product distribution of this example is summarised in Table 1.

### Example 2

### Ethylene Oligomerisation Reaction Using Cr(acetylacetonate)₃, (benzyl)₂PN(methyl)N(methyl)P(benzyl)₂ (Ligand 1b) and MMAO-3A in Cyclohexane at 60° C./5000 kPa

A solution of 1.36 mg of (benzyl)₂PN(methyl)N(methyl)P(benzyl)₂ (2.8 mmol) in 5 ml of cyclohexane was added to a solution of 0.9 mg Cr(acetylacetonate)₃ (2.5 mmol) in 5 ml cyclohexane in a Schlenk tube. MMAO-3A (modified methylaluminoxane, 2.4 mmol) was added and the mixture was immediately transferred to a 300 ml pressure reactor (autoclave) containing cyclohexane (90 ml) at 55° C. The autoclave was charged with ethylene after which the reactor temperature was controlled at 60° C., while the ethylene pressure was maintained at 5000 kPa. The reaction was terminated after 30 min and the work-up procedure of Example 1 above was employed. The total product mass was 11.35 g. The product distribution of this example is summarised in Table 1.

### Comparative Example 3 (Relative to Example 4 and Example 5)

### Ethylene Oligomerisation Reaction Using Cr(acetylacetonate)₃, (phenyl)₂PN(ethyl)N(ethyl)P(phenyl)₂ (Ligand 1c) and MMAO-3 in Methylcyclohexane at 60° C./4500 kPa

A solution of 1.14 mg of (phenyl)₂PN(ethyl)N(ethyl)P(phenyl)₂ (2.5 μmol) in 1.0 ml of methylcyclohexane was added to a solution of 0.88 mg chromium(acetylacetonate)₃ (2.5 μmol) in 1.0 ml of methylcyclohexane in a Schlenk tube. MMAO-3A (modified methylaluminoxane, 2.4 mmol) was added to this solution. This mixture was then transferred to a 450 ml pressure reactor (autoclave) at 55° C. containing methylcyclohexane (100 ml). The autoclave was charged with ethylene after which the reactor temperature was controlled at 60° C., while the ethylene pressure was maintained at 4500 kPa. The reaction was terminated after 18 min and the work-up procedure of Example 1 above was employed. The total product mass was 152.37 g. The product distribution of this example is summarised in Table 1.

### Example 4

### Ethylene Oligomerisation Reaction Using Cr(acetylacetonate)₃, (benzyl)₂PN(ethyl)N(ethyl)P(benzyl)₂ (Ligand 1d) and MMAO-3A in Cyclohexane at 60° C./5000 kPa

A solution of 1.43 mg of (benzyl)₂PN(ethyl)N(ethyl)P(benzyl)₂ (2.8 mmol) in 5 ml of cyclohexane was added to a solution of 0.9 mg Cr(acetylacetonate)₃ (2.5 μmol) in 5 ml cyclohexane in a Schlenk tube. MMAO-3A (modified methylaluminoxane, 2.4 mmol) was added and the mixture was immediately transferred to a 300 ml pressure reactor (autoclave) containing cyclohexane (90 ml) at 55° C. The autoclave was charged with ethylene after which the reactor temperature was controlled at 60° C., while the ethylene pressure was maintained at 5000 kPa. The reaction was terminated after 30 min by discontinuing the ethylene feed to the reactor and the work-up procedure of Example 1 above was employed. The total product mass was 37.76 g. The product distribution of this example is summarised in Table 1.

### Example 5

### Ethylene Oligomerisation Reaction Using Cr(acetylacetonate)₃, (allyl)₂PN(ethyl)N(ethyl)P(allyl)₂ (Ligand 1e) and MMAO-3A in Methylcyclohexane at 60° C./5000 kPa

A solution of 1.56 mg of (allyl)₂PN(ethyl)N(ethyl)P(allyl)₂ (5.0 μmol) in 2.0 ml of methylcyclohexane was added to a solution of 1.76 mg chromium(acetylacetonate)₃ (5.0 μmol) in 2.0 ml of methylcyclohexane in a Schlenk tube. MMAO-3A (modified methylaluminoxane, 4.8 mmol) was added to this solution. This mixture was then transferred to a 300 ml pressure reactor (autoclave) containing a 90 ml of methylcyclohexane at 55° C. The autoclave was charged with ethylene after which the reactor temperature was controlled at 60° C., while the ethylene pressure was maintained at 5000 kPa. The reaction was terminated after 30 min and the work-up procedure of Example 1 above was employed. The total product mass was 15.05 g. The product distribution of this example is summarised in Table 1.

### Comparative Example 6 (Relative to Examples 7 and 8)

### Ethylene Oligomerisation Reaction Using Cr(acetylacetonate)₃, (phenyl)₂PN(isopropyl)P(phenyl)₂ (ligand 2a) and MMAO-3A in Methylcyclohexane at 60° C./4500 kPa

A solution of 1.07 mg of (phenyl)₂PN(isopropyl)P(phenyl)₂ (2.5 mmol) in 1 ml of methylcyclohexane was added to a solution of 0.88 mg Cr(acetylacetonate)₃ (2.5 mmol) in 1 ml methylcyclohexane in a Schlenk tube. MMAO-3A (modified methylaluminoxane, 2.4 mmol) was added and the mixture was immediately transferred to a 300 ml pressure reactor (autoclave) containing methylcyclohexane (100 ml) at 55° C. The autoclave was charged with ethylene after which the reactor temperature was controlled at 60° C., while the ethylene pressure was maintained at 4500 kPa. The reaction was terminated after 23 min and the work-up procedure of Example 1 above was employed. The total product mass was 66.13 g. The product distribution of this example is summarised in Table 2.

### Example 7

### Ethylene Oligomerisation Reaction Using Cr(acetylacetonate)₃, (benzyl)₂PN(isopropyl)P(benzyl)₂ (ligand 2b) and MMAO-3A in Methylcyclohexane at 60° C./4500 kPa

A solution of 4.84 mg of (benzyl)₂PN(isopropyl)P(benzyl)₂ (10 μmol) in 4 ml of methylcyclohexane was added to a solution of 1.76 mg Cr(acetylacetonate)₃ (5 μmol) in 2 ml methylcyclohexane in a Schlenk tube. MMAO-3A (modified methylaluminoxane, 4.8 mmol) was added and the mixture was immediately transferred to a 300 ml pressure reactor (autoclave) at 55° C. containing 90 ml of methylcyclohexane. The autoclave was charged with ethylene after which the reactor temperature was controlled at 60° C., while the ethylene pressure was maintained at 4500 kPa. The reaction was terminated after 20 min and the work-up procedure of Example 1 above was employed. The total product mass was 51.02 g. The product distribution of this example is summarised in Table 2.

### Example 8

### Ethylene Oligomerisation Reaction Using Cr(acetylacetonate)₃, (phenyl)₂PN(isopropyl)P(phenyl)(CH₂CH₂-phenyl) (ligand 2c) and MMAO-3A in Methylcyclohexane at 60° C./4500 kPa

A solution of 4.98 mg of (phenyl)₂PN(isopropyl)P(phenyl)(CH₂CH₂-phenyl) (10 mmol) in 4 ml of methylcyclohexane was added to a solution of 1.76 mg Cr(acetylacetonate)₃ (5 μmol) in 2 ml methylcyclohexane in a Schlenk tube. MMAO-3A (modified methylaluminoxane, 4.8 mmol) was added and the mixture was immediately transferred to a 300 ml pressure reactor (autoclave) containing 90 ml of methylcyclohexane at 55° C. The autoclave was charged with ethylene after which the reactor temperature was controlled at 60° C., while the ethylene pressure was maintained at 4500 kPa. The reaction was terminated after 15 min and the work-up procedure of Example 1 above was employed. The total product mass was 1.39 g. The product distribution of this example is summarised in Table 2.

### Comparative Example 9 (Relative to Example 10)

### Preparation of the Complex {[(phenyl)₂P-1,2-phenylene-P(phenyl)₂]CrCl₃}₂ (Ligand 3a-CrC₃)

The complex {[(phenyl)₂P-1,2-phenylene-P(phenyl)₂]CrCl₃}₂ was prepared according to the synthetic procedure used for the preparation of [(phenyl)₂P)₂N(phenyl)CrCl₃]₂ as described in J. Am. Chem. Soc. 2004, 126, 14712.

### Ethylene Oligomerisation Reaction Using the Complex {[(phenyl)₂P-1,2-phenylene-P(phenyl)₂]CrCl₃}₂ and MMAO-3A in Cyclohexane at 80° C./5000 kPa

MMAO-3A (modified methylaluminoxane, 1.2 mmol) was added to a suspension of 1.51 mg of the complex {[(phenyl)₂P-1,2-phenylene-P(phenyl)₂]CrCl₃}₂ (1.25 mmol) and the mixture was immediately transferred to a 300 ml pressure reactor (autoclave) containing cyclohexane (90 ml) at 75° C. The autoclave was charged with ethylene after which the reactor temperature was controlled at 80° C., while the ethylene pressure was maintained at 5000 kPa. The reaction was terminated after 8.5 min and the work-up procedure of Example 1 above was employed. The total product mass was 63.53 g. The product distribution of this example is summarised in Table 3.

### Example 10

### Preparation of the Complex {[(benzyl)₂P-1,2-phenylene-P(benzyl)₂]CrCl₃}₂ (Ligand 3b-CrCl₃)

The complex {[(benzyl)₂P-1,2-phenylene-P(benzyl)₂]CrCl₃}₂ was prepared according to the synthetic procedure used for the preparation of [(phenyl)₂P)₂N(phenyl)CrCl₃]₂ as described in J. Am. Chem. Soc. 2004, 126, 14712.

### Ethylene Oligomerisation Reaction Using the complex {[(benzyl)₂P-1,2-phenylene-P(benzyl)₂]CrCl₃}₂ and MMAO-3A in Methylcyclohexane at 60° C./5000 kPa

MMAO-3A (modified methylaluminoxane, 1.92 mmol) was added to a suspension of 2.64 mg of the complex {[(benzyl)₂P-1,2-phenylene-P(benzyl)₂]CrCl₃}₂ (2 μmol) and the mixture was immediately transferred to a 300 ml pressure reactor (autoclave) containing methylcyclohexane (90 ml) at 55° C. The autoclave was charged with ethylene after which the reactor temperature was controlled at 60° C., while the ethylene pressure was maintained at 5000 kPa. The reaction was terminated after 12 min and the work-up procedure of Example 1 above was employed. The total product mass was 50.83 g. The product distribution of this example is summarised in Table 3.

### Comparative Example 11 (Relative to Example 12)

### Preparation of the Complex [(phenyl)₂P-1,2-phenylene-N═C(H)-cyclohexyl]CrCl₃ (Ligand 4a-CrCl₃)

The complex [(phenyl)₂P-1,2-phenylene-N═C(H)-cyclohexyl]CrCl₃ was prepared according to the synthetic procedure used for the preparation of [(phenyl)₂P)₂N(phenyl)CrCl₃] as described in J. Am. Chem. Soc. 2004, 126, 14712.

### Ethylene Oligomerisation Reaction Using the Complex [(phenyl)₂P-1,2-phenylene-N═C(H)-cyclohexyl]CrCl₃ and MMAO-3A in Methylcyclohexane at 60° C./4500 kPa

A suspension of 2.65 mg of [(phenyl)₂P-1,2-phenylene-N═C(H)-cyclohexyl]CrCl₃ (5 mmol) in 2 ml of methylcyclohexane was stirred overnight in a Schlenk tube. MMAO-3A (modified methylaluminoxane, 4.8 mmol) was added and the solution was transferred to a 300 ml pressure reactor (autoclave) containing methylcyclohexane (90 ml) at 55° C. The autoclave was charged with ethylene after which the reactor temperature was controlled at 60° C., while the ethylene pressure was maintained at 4500 kPa. The reaction was terminated after 20 min and the work-up procedure of Example 1 above was employed. The total product mass was 0.69 g. The product distribution of this example is summarised in Table 4.

### Example 12

### Preparation of the Complex [(phenyl)₂P-1,2-phenylene-N═C(H)-phenyl]CrCl₃ (Ligand 4b-CrCl₃)

The complex [(phenyl)₂P(1,2-phenylene)NC(H)-phenyl]CrCl₃ was prepared from Cr(THF)₃Cl₃ and the ligand according to the synthetic procedure used for the preparation of [(phenyl)I₂P)₂N(phenyl)CrCl₃]₂ as described in J. Am. Chem. Soc. 2004, 126, 14712.

### Ethylene Oligomerisation Reaction Using the Complex [(phenyl)₂P-1,2-phenylene-N═C(H)-phenyl]CrCl₃ and MMAO-3A in Methylcyclohexane at 60° C./4500 kPa

A suspension of 2.62 mg of [(phenyl)₂P-1,2-phenylene-N═C(H)-phenyl]CrCl₃ (5 mmol) in 2 ml of methylcyclohexane was stirred overnight in a Schlenk tube. MMAO-3A (modified methylaluminoxane, 4.8 mmol) was added and the solution was transferred to a 300 ml pressure reactor (autoclave) containing methylcyclohexane (90 ml) at 55° C. The autoclave was charged with ethylene after which the reactor temperature was controlled at 60° C., while the ethylene pressure was maintained at 4500 kPa. The reaction was terminated after 15 min and the work-up procedure of Example 1 above was employed. The total product mass was 2.41 g. The product distribution of this example is summarised in Table 4.

### Comparative Example 13 (Relative to Example 14)

### Preparation of the Complex {[(phenyl)₂P-ethylene-N═C(H)-isobutyl]CrCl₃)₂ (Ligand 5a-CrCl₃)

The complex ([(phenyl)₂P-ethylene-N═C(H)-isobutyl]CrCl₃}₂ was prepared from Cr(THF)₃Cl₃ and the ligand according to the synthetic procedure used for the preparation of [(phenyl)₂P)₂N(phenyl)CrCl₃]₂ as described in J. Am. Chem. Soc. 2004, 126, 14712.

### Ethylene Oligomerisation Reaction Using the Complex ([(phenyl)₂P-ethylene-N═C(H)-isobutyl]CrCl₃)₂ and MMAO-3A in Methylcyclohexane at 60° C./5000 kPa

A suspension of 8.88 mg of {[(phenyl)₂P-ethylene-N═C(H)-isobutyl]CrCl₃}₂ (20 μmol) in 10 ml of methylcyclohexane was transferred to a 300 ml pressure reactor (autoclave) containing methylcyclohexane (90 ml) and MMAO-3A (modified methylaluminoxane, 9.6 mmol) at 55° C. The autoclave was charged with ethylene after which the reactor temperature was controlled at 60° C., while the ethylene pressure was maintained at 5000 kPa. The reaction was terminated after 60 min and the work-up procedure of Example 1 above was employed. The product distribution of this example is summarised in Table 4.

### Example 14

### Preparation of the Complex {[(phenyl)₂P-ethylene-N═C(H)-phenyl]CrCl₃}₂ (Ligand 5b-CrCl₃)

The complex {[(phenyl)₂P-ethylene-N═C(H)-phenyl]CrCl₃}₂ was prepared from Cr(THF)₃Cl₃ and the ligand according to the synthetic procedure used for the preparation of [(phenyl)₂P)₂N(phenyl)CrCl₃]₂ as described in J. Am. Chem. Soc. 2004, 126(45), 14712.

### Ethylene Oligomerisation Reaction Using the Complex {[(phenyl)₂P-ethylene-N═C(H)-phenyl]CrCl₃}₂ and MMAO-3A in Methylcyclohexane at 60° C./5000 kPa

A suspension of 9.27 mg of {[(phenyl)₂P-ethylene-N═C(H)-phenyl]CrCl₃}₂ (20 μmol) in 10 ml of methylcyclohexane was transferred to a 300 ml pressure reactor (autoclave) containing a mixture of methylcyclohexane (90 ml) and MMAO-3A (modified methylaluminoxane, 9.6 mmol) at 55° C. The autoclave was charged with ethylene after which the reactor temperature was controlled at 60° C., while the ethylene pressure was maintained at 5000 kPa. The reaction was terminated after 60 min and the work-up procedure of Example 1 above was employed. The total product mass was 12.2 g. The product distribution of this example is summarised in Table 4.

TABLE 1

| | | | | | | | | | | Liquid Product selectivity | | | | | | | |
|---|---|---|---|---|---|---|---|---|---|---|---|---|---|---|---|---|---|
| | | | | | | | | | | | | Total | | | | Total | Total |
| | | | | | | | | | | | 1C6 in | 1C6 in | | 1C8 in | | liquid | solid |
| | | | | | | | | | | | C6 | liquid | | C8 | | prod- | prod- |
| | Li- | R | | Cr | MMAO | p | T | Efficiency | Activity | C6 | fraction | product | C8 | fraction | C10+ | uct | uct |
| | gand | 1 | R 2 | umol | eq | barg | ° C. | g/g Cr | g/g Cr/h | % | % | % | % | % | % | % | % |
| 1 | 1a | Me | Ph | 2.5 | 960 | 45 | 60 | 895857 | 1414511 | 30.4 | 82.6 | 25.1 | 62.8 | 99.4 | 6.5 | 98.6 | 1.4 |
| 2 | 1b | Me | CH2Ph | 2.5 | 960 | 50 | 60 | 87 300 | 174 700 | 85.4 | 98.6 | 84.2 | 7.2 | 100.0 | 6.2 | 84.1 | 15.9 |
| 3 | 1c | Et | Ph | 2.5 | 960 | 45 | 60 | 1172047 | 3836486 | 56.4 | 97.3 | 54.9 | 34.0 | 99.4 | 9.5 | 99.6 | 0.4 |
| 4 | 1d | Et | CH2Ph | 2.5 | 960 | 50 | 60 | 290 400 | 580 900 | 96.1 | 99.8 | 95.9 | 0.7 | 100.0 | 3.0 | 97.9 | 2.1 |
| 5 | 1e | Et | CH2CHCH2 | 5 | 960 | 50 | 60 | 57902 | 115804 | 71.5 | 91.3 | 65.3 | 21.5 | 49.8 | 6.3 | 98.9 | 1.1 |

TABLE 2

| | | | | | | | | | | Liquid Product selectivity | | | | | | | |
|---|---|---|---|---|---|---|---|---|---|---|---|---|---|---|---|---|---|
| | | | | | | | | | | | 1C6 | | | 1C8 | | | |
| | | | | | | | | | | | in | Total | | in | | Total | Total |
| | | | | | | | | | | | C6 | 1C6 in | | C8 | | liquid | solid |
| | | | | | | | | | | | frac- | liquid | | frac- | | prod- | prod- |
| | Li- | | | Cr | MMAO | p | T | Efficiency | Activity | C6 | tion | product | C8 | tion | C10+ | uct | uct |
| | gand | R 1 | R 2 | umol | eq | barg | ° C. | g/g Cr | g/g Cr/ h | % | % | % | % | % | % | % | % |
| 6 | 2a | Ph | Ph | 2.5 | 960 | 45 | 60 | 508723 | 1327102 | 19.3 | 76.6 | 14.8 | 71.6 | 99.0 | 8.5 | 99.4 | 0.6 |
| 7 | 2b | CH2Ph | CH2Ph | 5 | 960 | 45 | 60 | 196212 | 588636 | 94.4 | 97.9 | 92.4 | 1.5 | 100.0 | 3.6 | 77.8 | 22.2 |
| 8 | 2c | Ph | CH2CH2Ph | 5 | 960 | 45 | 60 | 5336 | 21344 | 38.0 | 86.8 | 33.0 | 45.9 | 95.4 | 7.2 | 55.9 | 44.1 |

TABLE 3

| | | | | | | | | | Liquid Product selectivity | | | | | | | |
|---|---|---|---|---|---|---|---|---|---|---|---|---|---|---|---|---|
| | | | | | | | | | | | Total | | | | | |
| | | | | | | | | | | 1C6 in | 1C6 in | | 1C8 in | | Total | Total |
| | Li- | | | | | | | | | C6 | liquid | | C8 | | liquid | solid |
| | gand | | Cr | MMAO | p | T | Efficiency | Activity | C6 | fraction | product | C8 | fraction | C10+ | product | product |
| | R 1 | R 1 | umol | eq | barg | ° C. | g/g Cr | g/g Cr/h | % | % | % | % | % | % | % | % |
| 9 | 3a | Ph | 2.5 | 480 | 50 | 80 | 488700 | 3449600 | 39.0 | 74.5 | 29.1 | 51.1 | 97.5 | 8.5 | 98.4 | 1.6 |
| 10 | 3b | CH2Ph | 4 | 480 | 50 | 60 | 244400 | 1221800 | 92.9 | 99.3 | 92.2 | 2.1 | 97.1 | 4.9 | 99.6 | 0.5 |

TABLE 4

| | | | | | | | | | Liquid Product selectivity | | | | | | | |
|---|---|---|---|---|---|---|---|---|---|---|---|---|---|---|---|---|
| | | | | | | | | | | | Total | | | | | |
| | | | | | | | | | | 1C6 in | 1C6 in | | 1C8 in | | Total | Total |
| | | | | | | | | | | C6 | liquid | | C8 | | liquid | solid |
| | | | Cr | MMAO | p | T | Efficiency | Activity | C6 | fraction | product | C8 | fraction | C10+ | product | product |
| | Ligand | R 1 | umol | eq | barg | ° C. | g/g Cr | g/g Cr/h | % | % | % | % | % | % | % | % |
| 11 | 4a | Cyclohexyl | 5 | 960 | 45 | 60 | 2639 | 7916 | 42.9 | 100.0 | 42.9 | 12.0 | 50.0 | 0.0 | 53.7 | 46.4 |
| 12 | 4b | Ph | 5 | 960 | 45 | 60 | 9274 | 37098 | 84.6 | 95.5 | 80.8 | 2.6 | 92.3 | 0.0 | 61.7 | 38.3 |
| 13 | 5a | Isobutyl | 20 | 480 | 50 | 60 | 0 | 0 | 0.0 | 0.0 | 0.0 | 0.0 | 0.0 | 0.0 | 0.0 | 0.0 |
| 14 | 5b | Ph | 20 | 480 | 50 | 60 | 12000 | 12000 | 65.3 | 96.0 | 62.7 | 2.1 | 90.0 | 2.5 | 71.3 | 28.7 |

1. A process for trimerization of olefins wherein an olefinic feedstream is contacted with a catalyst system which includes the combination of
i) a source of a chromium; and
ii) a ligating compound of the formula
(R¹)ₘX¹(Y)X²(R²)ₙ
wherein:
X¹ and X² are independently an atom selected from the group consisting of N, P, As, Sb, Bi, O, S and Se or said atom oxidized by S, Se, N or O, where the valence of X¹ and/or X² allows for such oxidation;
Y is a linking group between X¹ and X²;
m and n are independently 0, 1 or a larger integer; and
R¹ and R² are independently selected from the group consisting of hydrogen, a hydrocarbyl group, a heterohydrocarbyl group, and an organoheteryl group; R¹ being the same or different when m>1; R² being the same or different when n>1; and at least one R¹ or R² is a moiety of formula
(L)(D)
wherein:
L is a linking moiety between X¹ or X² and D; wherein L is a hydrocarbon moiety selected from the group of hydrocarbon moieties consisting of moieties which include one or more carbon atoms where all carbon atoms only have saturated bonds, —CH₂—, hydrocarbon moieties which have one or more carbons with unsaturated bonds and ═CH— and D is an electron donating moiety which includes at least one multiple bond between adjacent atoms which multiple bond renders D capable of bonding with the chromium in the source of chromium; provided that when D is a moiety derived from an aromatic compound with a ring atom of the aromatic compound bound to L, D has no electron donating moiety that is bound to a ring atom of the aromatic compound adjacent to the ring atom bound to L, and that is in the form of a heterohydrocarbyl group, a heterohydrocarbylene group, a heterohydrocarbylidene group, or an organoheteryl group that is capable of bonding by a coordinate covalent bond to the chromium in the source of chromium,

2. The process as claimed in claim 1, wherein the trimerization process comprises the trimerization of a single monomer olefinic compound.

3. The process as claimed in claim 1, wherein the olefinic compound is selected from the group consisting of ethylene, propene, 1-butene, 1-pentene, 1-hexene, 1-heptene, and 1-octene, 1-nonene, 1-decene, 3-methyl-1-butene, 3-methyl-1-pentene, 4-methyl-1-pentene, styrene, p-methyl styrene, 1-dodecene and combinations thereof.

4. The process as claimed in claim 1, wherein the catalyst further includes one or more activators.

5. The process as claimed in claim 4, wherein the activator is selected from the group consisting of aluminium compounds, boron compounds, organic salts, inorganic acids and salts selected from the group consisting of tetrafluoroboric acid etherate, silver tetrafluoroborate, and sodium hexafluoroantimonate.

6. The process as claimed in claim 5, wherein the activator is selected from alkylaluminoxanes such as methylaluminoxane (MAO), high stability methylaluminoxane (MAO HS), and modified alkylaluminoxanes such as modified methylaluminoxane (MMAO).

7. The process as claimed in claim 1, wherein the source of chromium is selected from the group consisting of chromium trichlorlde tris-tetrahydrofuran; (benzene)trlcarbonyl chromium; chromium (III) octanoate; chromium (III) hexaonate; chromium hexacarbonyl; chromium (III) acetylacetonate, chromium (III) naphthenate, and chromium (III) 2-ethylhexanoate.

8. The process as claimed in claim 1, wherein D is a hydrocarbyl or a heterohydrocarbyl moiety which includes at least one multiple bond between adjacent atoms wherein at least one such multiple bond renders D capable of bonding by a coordinate covalent bond to the chromium.

9. The process of claim 8, wherein D is a substituted phenyl wherein one or more moieties other than H are bound as a non-ring atom to a ring atom of D.

10. The process as claimed in claim 9, wherein D is an aromatic moiety or heteroaromatic moiety selected from the group consisting of phenyl, naphthyl, 7-(1,2,3,4-tetrahydronaphthyl), anthracenyl, phenanthrenyl, phenalenyl, 3-pyridyl, 3-thiopeneyl, 7-benzofuranyl, 7-(2H-1-benzopyranyl), 7-quinolinyl and 8-benzisoxazolyl.

11. The process as claimed in claim 1, wherein L is bound to a single atom of D where D is an aromatic or a heteroaromatic moiety.

12. The process as claimed in claim 11, wherein L is bound to an atom of D which atom of D is linked to another atom of D by means of a multiple bond.

13. The process as claimed in claim 1, wherein L is bound to X¹ or X² by means of a single bond.

14. The process as claimed in claim 1, wherein L is bound to X¹ or X² by means of a double bond.

15. The process according to claim 1, wherein L is selected from —CH₂—, —CH═, —CH₂—CH₂—, —CH═CH—, —CH₂—CH₂—CH₂—, —CH═CH—CH₂—, —CH₂—CH═CH—, —CH(CH₃)—CH₂—CH₂—, —CH₂—CH(CH₃)—CH₂—, —CH₂—CH₂—CH(CH₃)— and —CH₂—C(CH₃)₂—CH₂—.

16. The process as claimed in claim 1, wherein (L)(D) is a moiety selected from benzyl, ethyl-phenyl, propyl-phenyl, methyl-naphthyl, ethyl-naphthyl, propyl-naphthyl, methyl-anthracenyl, methyl-phenanthrenyl, methyl-phenalenyl, methyl-3-(pyridyl), methyl-3-(thiopeneyl), methyl-7-(benzofuranyl), methyl-7-(2H-1-benzopyranyl), methyl-7-(quinolinyl) and methyl-6-(benzisoxazolyl).

17. The process as claimed in claim 1, wherein Y is selected from the group consisting of an organic linking group such as a hydrocarbylene, substituted hydrocarbylene, heterohyd rocarbylene and a substituted heterohydrocarbylene; an inorganic linking group comprising either a single- or two- atom linking spacer; and a group comprising; methylene; dimethylmethylene; ethylene; ethene-1,2-diyl; propane-1,2-diyl, propane-1,3-diyl; cyclopropane-1,1-diyl; cyclopropane-1,2-diyl; cyclobutane-1,2-diyl, cyclopentane-1,2-diyl, cyclohexane-1,2-diyl, cyclohexane-1,1-diyl; 1,2-phenylene; naphthalene-1,8-diyl; phenanthrene-9,10-diyl, phenanthrene-4,5-diyl, 1,2-catecholate, 1,2-diarylhydrazine-1,2-diyl (—N(Ar)—N(Ar)—) where Ar is an aryl group; 1,2-dialkylhydrazine-1,2-diyl (—N(Alk)—N(Alk)—) where Alk is an alkyl group; —B(R⁷)—, —Si(R⁷)₂—, —P(R⁷)— and —N(R⁷)—where R⁷ is hydrogen, a hydrocarbyl, a heterohydrocarbyl, a organoheteryl or halogen.

18. The process as claimed in claim 17, wherein Y is —N(R⁷)— and R⁷ is selected from the group consisting of hydrogen, alkyl, substituted alkyl, aryl, substituted aryl, aryloxy, substituted aryloxy, halogen, alkoxycarbonyl, carbonyloxy, alkoxy, aminocarbonyl, carbonylamino, dialkylamino, silyl groups or derivatives thereof, and aryl substituted with any of these substituents.

19. The process as claimed in claim 18, wherein Y is —N(R⁷)— and R⁷ is selected from the group consisting of methyl, ethyl, propyl, isopropyl, cyclopropyl, allyl, butyl, tertiary-butyl, sec-butyl, cyclobutyl, pentyl, isopentyl, 1,2-dimethylpropyl (3-methyl-2-butyl), 1,2,2-trimethylpropyl (R/S-3,3-dimethyl-2-butyl), 1-(1-methylcyclopropyl)-ethyl, neopentyl, cyclopentyl, cyclohexyl, cycloheptyl, cyclo-octyl, decyl, cyclodecyl, 1,5-dimetylheptyl, 2-naphthylethyl, 1-naphthylmethyl, adamantylmethyl, 1-adamantyl, 2-adamantyl, 2-isopropylcyclohexyl, 2,6-dimethylcyclohexyl, cyclododecyl, 2-methylcyclohexyl, 3-methylcyclohexyl, 4-methylcyclohexyl, 2-ethylcyclohexyl, 2-isopropylcyclohexyl, 2,6-dimethyl-cyclohexyl, exo-2-norbornanyl, isopinocamphenyl, dimethylamino, phthalimido, pyrrolyl, trimethylsilyl, dimethyl-tertiary-butylsilyl, 3-trimethoxylsilane-propyl, indanyl, cyclohexanemethyl, 2-methoxyphenyl, 3-methoxyphenyl, 4-methoxyphenyl, 4-tertiary-butyiphenyl, 4-nitrophenyl, (1,1′-bis(cyclohexyl)-4,4′-methylene), 1,6-hexylene, 1-naphthyl, 2-naphthyl, N-morpholine, diphenylmethyl, 1,2-diphenyl-ethyl, phenylethyl, 2-methyiphenyl, 3-methylphenyl, 4-methylphenyl, 2,6-dimethyl-phenyl, 1,2,3,4-tetrahydronapththyl, or a 2-octyl group.

20. The process as claimed in claim 1, wherein Y is a moiety of formula
—Y¹—Y²—
wherein: Y¹ and Y² are independently CR₂¹⁹ or AR²⁰, wherein R¹⁹ and R²⁰ are independently hydrogen, a hydrocarbyl group or a heterocyclocarbyl group, and A is selected from the group consisting of N, P, As, Sb and Bi.

21. The process as claimed in claim 24, wherein Y is 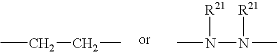
wherein each R²¹ is independently a hydrocarbyl group.

22. The process as claimed in claim 21, wherein R²¹ is an alkyl group.

23. The process as claimed in claim 1, wherein Y comprises a moiety derived from a cyclic compound wherein two atoms of the cyclic ring structure are bonded to X¹ and X² respectively.

24. The process as claimed in claim 1, wherein at least one of X¹ and X² is a potential electron donor for coordination with the transition metal referred to in (i)

25. The process as claimed in claim 1, wherein the ligating compound is of the formula 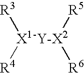
wherein
Y is a linking group between X¹ and X²; X¹ and X² are independently an atom selected from the group consisting of N, P, As, Sb and Bi or said atom oxidized by S, Se, N or O, where the valence of X¹ and/or X² allows for such oxidation; and R³ to R⁶ are each independently hydrogen, a hydrocarbyl group, a heterohydrocarbyl group, or organoheteryl group and at least one of R³ to R⁶ is a moiety of formula
(L)(D)
wherein:
L is a linking moiety between X¹ or X² and D; and
D is an electron donating moiety which includes at least one multiple bond between adjacent atoms which multiple bond renders D capable of bonding with the transition metal in the source of transition metal;
provided that when D is an aromatic compound with a ring atom of the aromatic compound bound to L, D has no electron donating moiety that is bound to a ring atom of the aromatic compound adjacent to the ring atom bound to L, and that is in the form of a heterohydrocarbyl group, a heterohydrocarbylene group, a heterohydrocarbylidene group, or an organoheteryl group that is capable of bonding by a coordinate covalent bond to the transition metal in the source of transition metal.

26. The process as claimed in claim 1, wherein X¹ or X² are both P.

27. The process as claimed in claim 1, wherein the ligating compound is of the formula 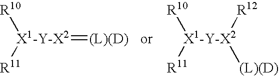
wherein:
Y is a linking group between X¹ and X²;
L is a linking moiety between X² and D; and
D is an electron donating moiety which includes at least one multiple bond between adjacent atoms which multiple bond renders D capable of bonding with the transition metal in the source of transition metal; provided that when D is a moiety derived from an aromatic compound with a ring atom of the aromatic compound bound to L, D has no electron donating moiety that is bound to a ring atom of the aromatic compound adjacent to the ring atom bound to L, and that is in the form of a heterohydrocarbyl group, a heterohydrocarbylene group, a heterohydrocarbylidene group, or an organoheteryl group that is capable of bonding by a coordinate covalent bond to the transition metal in the source of transition metal; X¹ or X² are independently an atom selected from the group consisting of N, P, As, Sb and Bi or said atom oxidized by S, Se, N or O, where the valence of X¹ and/or X² allows for such oxidation; R¹⁰ to R¹² are each independently hydrogen, a hydrocarbyl group, a heterohydrocarbyl group or an organoheteryl group.

28. The process as claimed in claim 31, wherein 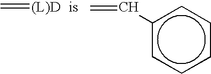
and -(L)(D) is benzyl.

29. The process as claimed in claim 1, wherein the ligating compound is selected from the group consisting of
(benzyl)₂PN(methyl)N(methyl)P(benzyl)₂;
(benzyl)₂PN(ethyl)N(ethyl)P(benzyl)₂;
(benzyl)₂PN(i-propyl)N(i-propyl)P(benzyl)₂;
(benzyl)₂PN(methyl)N(ethyl)P(benzyl)₂;
(benzyl)₂PN(methyl)N(i-propyl)P(benzyl)₂;
(benzyl)₂PN(methyl)N(t-butyl)P(benzyl)₂;
(benzyl)₂PCH₂N(i-propyl)P(benzyl)₂;
(allyl)₂PN(ethyl)N(ethyl)P(allyl)₂;
(phenyl)₂P—C₂H₄—N═C(H)-phenyl;
(phenyl)₂P—C₂H₄—N(H)—CH₂-phenyl;
(benzyl)(phenyl)PN(ethyl)N(ethyl)P(benzyl)(phenyl);
(benzyl)(phenyl)PN(ethyl)N(ethyl)P(phenyl)₂;
(benzyl)(phenyl)PN(ethyl)N(ethyl)P(benzyl)₂;
(ethyl-phenyl)₂PN(ethyl)N(ethyl)P(ethyl-phenyl)₂;
(propyl-phenyl)₂PN(ethyl)N(ethyl)P(propyl-phenyl)₂;
(methyl-naphthyl)₂PN(ethyl)N(ethyl)P(methyl-naphthyl)₂;
(ethyl-naphthyl)₂PN(ethyl)N(ethyl)P(ethyl-naphthyl)₂;
(benzyl)₂PN(isopropyl)P(benzyl)₂;
(benzyl)₂PN(methyl)P(benzyl)₂;
(benzyl)₂PN(ethyl)P(benzyl)₂;
(benzyl)₂PN(1,2-dimethylpropyl)P(benzyl)₂;
(benzyl)₂P-ethene-1,2-diyl-P(benzyl)₂;
(benzyl)₂P-ethylene-P(benzyl)₂;
(benzyl)₂P-1,2-phenylene-P(benzyl)₂.

30. The process as claimed in claim 1, wherein the ligating compound includes a polymeric moiety.

31. The process as claimed in claim 1, wherein the reaction is carried out in an inert solvent.

32. An trimerization product prepared by a process according to claim 1.

33. An oligomerisation catalyst which includes the combination of
i) a source of a transition metal; and
ii) a ligating compound of the formula
(R¹)ₘX¹(Y)X²(R²)ₙ
wherein:
X¹ and X² are independently selected from the group consisting of N, P, As, Sb, Bi, O, S and Se;
Y is a linking group between X¹ and X²;
m and n are independently 0, 1 or a larger integer; and
R¹ and R² are independently selected from the group consisting of hydrogen, a hydrocarbyl group, a heterohydrocarbyl group an organoheteryl group; R¹ being the same or different when m>1; R² being the same or different when n>1; and at least one R¹ or R² is a moiety of formula
(L)(D)
wherein:
L is a linking moiety between X¹ or X² and D; and
D is an electron donating moiety which includes at least one multiple bond between adjacent atoms which multiple bond renders D capable of bonding with the transition metal in the source of chromium; provided that when D is a moiety derived from an aromatic compound with a ring atom of the aromatic compound bound to L, D has no electron donating moiety that is bound to a ring atom of the aromatic compound adjacent to the ring atom bound to L, and that is in the form of a heterohydrocarbyl group, a heterohydrocarbylene group, a heterohydrocarbylidene group, or an organoheteryl group that is capable of bonding by a coordinate covalent bond to the transition metal in the source of chromium.
